(19) United States
(12) Patent Application Publication
(10) Pub. No.: US 2024/0245831 A1
(43) Pub. Date: Jul. 25, 2024

(54) Development of decellularized bovine bone graft

(71) Applicant: University of Sharjah, Sharjah (AE)

(72) Inventors: AB Rani SAMSUDDIN, Sharjah (AE); Sausan ALKAWAS, Sharjah (AE); Ali AbdulQadir ALQABBANI, Sharjah (AE); Aghila RANI K.G, Sharjah (AE)

(21) Appl. No.: 18/098,994

(22) Filed: Jan. 19, 2023

(51) Int. Cl. A61L 27/36

(52) U.S. Cl. CPC A61L 27/3687; A61L 27/3608; A61L 27/365; A61L 27/3691; A61L 2430/40

(57) ABSTRACT

Novel process for decellularization of bovine bone graft for reconstruction of bone defects.

### TECHNICAL FIELD

The present invention relates to novel method for decellularization of bovine bone graft.

### BACKGROUND

Bovine bone grafts are well-documented in literature and by far the most commonly used xenografts in dentistry [1]. It is deproteinized by heating to eliminate the risk of allergic reactions and disease transmission factors and is used for developing grafts. The removal of proteins transforms it into biologically derived hydroxyapatite ceramic with well-preserved 3D natural bone structure similar to human bone. The trabecular architecture with interconnecting pores allows for optimal in-growth allowing the development of new vascularity. Guided osseous integration leads to excellent volume stability of the graft and favors formation of new bone on the highly structured bovine bone surface [2]. The bovine bone xenograft is osteoconductive and is available in a range of volumes and particle sizes.

Bone is the second most common transplant tissue after blood. Bone transplantation induces osteogenesis to repair bone defects. Allogenic bone transplantation and synthetic graft substitutes are efficient substitutes for transplantation but associated with several risks such as insufficient tissue healing, or secondary damage to the donor site and cellular and humoral immune reactions. Synthetic grafts additionally lack osteoinductive or osteogenic properties and have various effects on bone healing [3]. Decellularization enabled the development of natural biocompatible three-dimensional xenografts with limited cytotoxicity and immunoreactivity for the reconstruction of bone defects [4]. Decellularization is essential for eradicating cellular components and antigenicity from the bone tissue, thus avoiding disease transmission and inflammatory immune responses following graft implantation. This reduces the chances of failure and rejection of graft by the human or animal [5].

In the decellularization process, chemical and enzymatic, physical, or combinative methods are utilized to remove cells and DNA from the tissue while preserving its structural and regulatory proteins [5]. Currently, there are various methods of decellularization is followed, whether by chemicals or enzymes or the uses of both like DNAse, RNAse, EGTA, Triton X 100 or Sodium dodecyl sulfate (SDS) or Sodium deoxycholate (SD). Sodium dodecyl sulfate (SDS) is an ionic surfactant and possesses cytotoxic properties which has the ability to meet the standard requirements of complete cell removal and elimination of at least 90% of host DNA in several types of tissues and organs and require excessive thorough {washing by Triton X 100 which is nonionic to remove the remnant of SDS. This decellularization method aims to remove as much cellular material and at the same time preserving the native architecture of the processed bone. However, it is believed DNA and RNA still remain, and that may explain the occasional chronic inflammatory response occurring in some cases [6, 7]. Recent studies have shown the efficacy of new chemico-enzymatic protocol in the preparation of the bovine bone based on the osmotic shock which enhances the removal of cell component, biocompatibility, host tissue reaction and the absence of any inflammatory reaction in host tissues and preservation of the extracellular matrix structure of the bovine bone [6]. According to some authors, Sodium deoxycholate (SD) is another ionic surfactant that works by solubilizing the cell membrane. Unlike SDS, SD-produced scaffolds that were highly biocompatible, as cells seeded on the SD-decellularized matrices exhibited higher metabolic activity compared to those decellularized via SDS. A combined method of decellularization would be an ideal choice as the methods/factors used to complement one another with the goal of retaining desired characteristics in the engineered tissue free of DNA. For example, mechanical methods are typically less damaging to the tissue's structure; however, they fail to meet the requirements for immunogenicity. On the other hand, surfactants at low concentrations or enzymes used alone may not completely remove all cellular debris. In this regard, combining the physicochemical treatments and the enzymatic methods at optimum concentrations in a multistep process can yield an appropriate decellularized ECM with limited cytotoxicity and immunoreactivity in clinical applications. [6, 8].

Additionally, Muslim Community would seek bone graft that is from bovine “Halal” source instead of bone grafts from “Porcine (pig)” source or form other Non-Halal source. Therefore, there is a need for biocompatible and immunocompatible grafts suitable to be used as scaffolds for bone replacement that are derived from decellularized bovine bone.

### SUMMARY

In one embodiment of the present disclosure, disclosed herein is a process for the decellularization of a bovine bone scaffold, including:
(a) washing a cancellous bone body with a distilled water under pressure
(b) immersing the cancellous bone body in a mixture of alcohol-chloroform at a 1:1 ratio for approximately 24 hr with gentle shaking at the speed of approximately 25 to 75 rpm at room temperature;
(c) rinsing the cancellous bone body in deionized water at least three times;
(d) placing the cancellous bone body in distilled water under rotation at approximately 50 to 100 rpm for a time range of approximately 12 to 36 hr;
(e) deproteinizing the cancellous bone body in approximately 4% sodium hypochlorite for a time range of approximately 18 to 30 hr at room temperature;
(f) rinsing the cancellous bone body in a processing solution;
(g) washing the cancellous bone body sequentially in 0.01, 0.1, and 1% sodium dodecyl sulfate for approximately 72 hr;
(h) treating the cancellous bone body with a non-ionic detergent;
(i) treating the cancellous bone body with DNase and RNase for approximately a week, wherein at the end of the treatment the cancellous bone body is decellularized;
(j) rinsing the decellularized cancellous bone body with deionized water for approximately 3 hr;
(k) cryopreserving the decellularized cancellous bone block;
(l) lyophilizing the decellularized cancellous bone body;
(m) sterilizing the decellularized cancellous bone body utilizing gamma radiation.

In one aspect of the present disclosure, the cancellous bone body is a cancellous femoral head.

In one aspect of the present disclosure, the cancellous bone body is fragmented in blocks.

In one aspect of the present disclosure, the cancellous bone body is washed with a high-pressure water-jet spray that has a pressure equal or higher to 160 psi.

In a preferred aspect of the present disclosure, the cancellous bone body is immersed in a mixture of alcohol-chloroform at a 1:1 ratio for approximately 24 hr with gentle shaking at the speed of approximately 50 rpm at room temperature.

In a preferred aspect of the present disclosure, the cancellous bone body is placed in distilled water under continuous rotation at approximately 80 rpm for approximately 24 hr.

In a preferred aspect of the present disclosure, the cancellous bone body is deproteinized in 4% sodium hypochlorite for approximately 24 hr.

In a preferred aspect of the present disclosure, processing solution is a buffer.

In a most preferred aspect of the present disclosure, the buffer is phosphate-buffered saline.

In a preferred aspect, the non-ionic detergent is 1% Triton X-100.

In a preferred aspect of the present disclosure, the DNase and RNase are replaced every 12 hr with freshly prepared DNase and RNase.

In a preferred aspect of the present disclosure, the cancellous bone body is immersed in phosphate-buffered saline at 37° C. under continuous shaking when treated with DNase and RNase.

In one aspect of the present disclosure, the decellularized cancellous bone body contains less than approximately 50 ng dsDNA per mg ECM dry weight.

In a preferred aspect of the present disclosure, the decellularized cancellous bone body contains less than approximately 10 ng dsDNA per mg ECM dry weight.

In one aspect of the present disclosure, the decellularized cancellous bone body contains less than 200 bp DNA long fragments.

In one aspect of the present disclosure, the decellularized cancellous bone body does not have visible nuclear material by Hematoxylin and Eosin staining.

In a preferred aspect of the present disclosure, the cryopreservation method utilized is freeze-drying.

In one aspect of the present disclosure, the level of endotoxin in the decellularized cancellous bone body is less than 0.05 endotoxin units per mL.

In one aspect of the present disclosure, the level of endotoxin in the decellularized cancellous bone body is less than 0.02 endotoxin units per mL.

### BRIEF DESCRIPTION OF THE DRAWINGS

The accompanying drawings are not intended to be drawn to scale. In the drawings, each identical or nearly identical component that is illustrated in various figures is represented by a like numeral. For purposes of clarity, not every component may be labeled in every drawing. In the drawings:

FIG. 1 provides a bone processing steps. Schematic diagram depicting the sequential processing steps of producing lyophilized scaffolds of demineralized bovine cancellous bone (DMB) and decellularized bovine cancellous bone (DCC).

FIG. 2 provides: (A) a gross appearance of DMB and DCC scaffolds. The DMB and DCC scaffolds appeared as a clean white sponge, with gross porous structure compared to untreated native control bone. (B) Histology of control native bone (i, iv), DMB (ii, v), and DCC (iii, vi) scaffolds. Staining with Hematoxylin and Eosin showing intact nuclei in control native bone while nuclei were largely removed from the cells in the DMB scaffold and were entirely absent in the DCC scaffold resulting in empty lacunae with no visibly-stained nucleus within the lacunae (yellow arrow). The scale bar is 50 μm (upper panel) and 20 μm (bottom panel). (C) Representative SEM images of DMB and DCC scaffolds compared to native control. Magnification at 100×, 1000× and 5000× is indicated. (D) EDS surface analysis of scaffolds. Representative spectrographic analysis by EDS surface analysis technique showing the surface chemical elements detected in DMB and DCC scaffolds compared to native control.

FIG. 3 provides Variations in pore morphology related to scaffold treatment. Representative images showing pore sizes on the surface of DMB and DCC scaffold. Mean pore sizes of DMB (a) and DCC (b) scaffolds. DCC scaffolds present significantly wider pore diameter with richer interconnectivity compared to DMB at 100× magnification (c) (*p 0.05).

FIG. 4 provides: (a) lipid quantification in scaffolds. Quantification of residual lipids by Oil Red O staining in DMB and DCC scaffolds compared to native control. Values are expressed as mean±SEM (n=5). Statistically significant difference of *p<0.05. (b) Quantification of total collagen content in DMB and DCC scaffolds compared to native control bone. A significant reduction in total collagen content is observed in DMB and DCC scaffolds compared to native control. Values are expressed as mean±SEM (n=5; *p<0.05). The reduction in collagen content is more significant in DCC than DMB treatment (*p<0.05).

FIG. 5 provides FTIR spectra of DMB and DCC scaffolds compared to native control. A broad absorption peak of cholesterol is evident at 3412 cm-1 in native control bone, whereas a similar peak is absent in DMB and DCC scaffolds (a). FTIR Spectrum of DMB and DCC bone scaffolds compared to native control bone. Regions of interest are indicated: carbonate (850-890 cm-1), phosphate (900-1200 cm-1), amide I (1585-1720 cm-1), amide II (1500-1600-1), and amide III (1250-1350 cm-1) absorption peaks (b).

FIG. 6 provides quantification of the total nucleic acids in DCC scaffolds. The amount of residual DNA (a) and RNA (b) in the DCC scaffolds compared to native control bone is shown. Total residual DNA and RNA obtained were normalized by the dry weight of each bone sample and interpreted. Values are expressed as mean±SEM (n=5). Statistically significant differences of *p<0.0001 and **p<0.01 are indicated. (c) Agarose gel electrophoresis image of the total DNA extracted from DMB scaffold and day 0, 1, 4, and 7 of post DNase treatment of DCC scaffolds compared to native control bone.

FIG. 7 provides stress-strain curve of scaffolds in the compression test. Representative images of compressive engineering stress and engineering strain (SS) curves of native control bone (a), DMB (b), and DCC (c) scaffolds.

FIG. 8 provides stress-strain curve of scaffolds in three-point bending test. Representative images of flexural stress and strain curves of native control bone (a), DMB (b), and DCC (c) scaffolds.

FIG. 9 provides (a-b) Cell viability study employing DMB and DCC scaffolds by direct method (a) and indirect method (b). CM: complete media. (c) HOB cells seeded on DMB and DCC scaffolds showing optimum cell attachment by DAPI staining. (i) and (iii) represent DMB scaffolds and (ii) and (iv) represent DCC scaffolds captured by fluorescence and confocal microscopy respectively. (d-e) Scanning electron micrograph of HOB cells seeded onto DMB (d) and DCC (e) scaffolds. The osteoblast shows a healthy morphology residing on the ECM with evidence of filopodia spread.

FIG. 10 provides photomicrographs of HOB cells stained with Alizarin Red S showing increased formation of mineralized nodules in osteogenic medium (OS+) than without supplements (OS−). More significant mineralization was seen by day 14 (a). Alizarin Red S stained mineralized nodules quantification of human osteoblast cells seeded on DMB and DCC scaffolds grown in the presence or absence of osteogenic induction media at day 7 and 14 (b). The absorbance of the extracted solution measured at 405 nm. Data presented as mean±SEM (n=6). Quantification of Alizarin Red S staining in osteoblast cell seeded DMB and DCC scaffolds on day 14 in the presence and absence of osteogenic supplements (c). Data presented as mean±SEM.

FIG. 11 provides gene expression during mineralization of human osteoblast cells grown on DMB and DCC scaffolds in osteogenic medium for 14 days. Expression of genes was analyzed by real-time PCR. GAPDH expression was used as internal control.

FIG. 12 shows the final product of gamma sterilized decellularized bovine bone graft.

FIG. 13 illustrates the process of product development of decellularized bovine bone graft.

### DEFINITIONS

As used in this description and the accompanying claims, the following terms shall have the meanings indicated, unless the context otherwise requires:

As used herein, the singular forms “a, an” and “the” include plural references unless the content clearly dictates otherwise.

To the extent that the term “include,” “have,” or the like is used in the description or the claims, such term is intended to be inclusive in a manner similar to the term “comprise” as “comprise” is interpreted when employed as a transitional word in a claim.

The word “exemplary” is used herein to mean “serving as an example, instance, or illustration.” Any embodiment described herein as “exemplary” is not necessarily to be construed as preferred or advantageous over other embodiments.

The term “scaffold” in accordance with the present invention, includes scaffold, body, block, chip, substrate, matrix, or segment.

Unless defined otherwise, all technical and scientific terms used herein have the same meaning as commonly understood by one of ordinary skill in the art to which the disclosure belongs. Although any methods and materials similar to or equivalent to those described herein may be used in the practice or testing of the present disclosure, the preferred materials and methods are described below.

### DETAILED DESCRIPTION

A study was conducted to identify a novel decellularization process for bovine bone scaffolds (DCC) that would ensure minimal damage to the bone and thus producing high quality bone grafts with high chance of success in reconstructive surgical operations. Recent advances in tissue engineering have led to potential new strategies for the repair, replacement and regeneration of bone defects. Cancellous bovine bone represents an inexhaustible source material for bone tissue engineering and using the methods of decellularization, natural, biocompatible, three dimensional grafts have been developed for reconstruction of bone defects. However, removal of the genetic material/DNA from the bone matrix is critical because the presence of graft DNA may stimulate deleterious immune reactions in the host by activating cytokine production and humoral responses. An ideal bone graft must have complete removal of the graft cellular and genetic materials without compromising the structural integrity of the extra cellular matrix.

In a first aspect, the novel method of decellularization of the present disclosure employs physicochemical and enzymatic treatments in order to develop biocompatible and immunocompatible grafts that are suitable to be used as scaffolds for bone replacement and bone regeneration. Bovine bones obtained locally and processed by decellularization followed by lyophilization and gamma sterilization. The bone grafts will be characterized for the presence of total DNA, collagen and glycosaminoglycan content and structural features assessed by histology and SEM.

In a second aspect, the bovine femoral head was chosen as the source of starting material, due to its spongy cancellous structure and composition that is suitable in dental applications. The presence of marrow spaces within the structure of cancellous bone scaffold, and its large surface area, makes it very attractive to graft sites requiring less mechanical strength, and as space filler mostly preferred for repairing dental defects.

In a third aspect, fundamental goal of any decellularization protocol is to remove all cellular material without adversely affecting the composition, integrity, mechanical property, and eventual biological activity of the remaining ECM. It is essential for the implanted grafts to favor a mechanically stable interface with fusion of the implant surface and the bone tissue. The main requirements for bone grafts are osteoconduction (new bone growth on the graft), osteoinduction (cells differentiating into bone forming osteoblasts) and osteogenesis (bone/callus formation) [9]. The decellularized cancellous bovine bone scaffold of the present disclosure enables the promotion of cell adhesion on the osteoconductive material in order to create an osteoinductive graft.

In a fourth aspect, osteogenesis entails cellular activity by osteoblasts, osteocytes and osteoclasts. Osteoblasts possess signaling molecules and act as mechanosensory cells. Hence it is important to investigate the adhesion and proliferation behavior of human osteoblast-like cells over time when incubated on surfaces of the graft materials. Cell attachment is linked to cell viability through integrin binding and may enhance the survival of implanted cells [10]. The decellularized cancellous bovine bone scaffold of the present disclosure supports the growth and proliferation of osteoblast cells.

In another aspect, the novel decellularization process of the present disclosure was used to produce bovine cancellous bone scaffold and its physicochemical, mechanical, and biological characteristics were compared in-vitro with demineralized cancellous bone scaffold.

In one embodiment of the present disclosure, disclosed herein is a process for the decellularization of a bovine bone scaffold, including:
(n) washing a cancellous bone body with a distilled water under pressure
(o) immersing the cancellous bone body in a mixture of alcohol-chloroform at a 1:1 ratio for approximately 24 hr with gentle shaking at the speed of approximately 25 to 75 rpm at room temperature;
(p) rinsing the cancellous bone body in deionized water at least three times;
(q) placing the cancellous bone body in distilled water under rotation at approximately 50 to 100 rpm for a time range of approximately 12 to 36 hr;
(r) deproteinizing the cancellous bone body in approximately 4% sodium hypochlorite for a time range of approximately 18 to 30 hr at room temperature;
(s) rinsing the cancellous bone body in a processing solution;
(t) washing the cancellous bone body sequentially in 0.01, 0.1, and 1% sodium dodecyl sulfate for approximately 72 hr;
(u) treating the cancellous bone body with a non-ionic detergent;
(v) treating the cancellous bone body with DNase and RNase for approximately a week, wherein at the end of the treatment the cancellous bone body is decellularized;
(w) rinsing the decellularized cancellous bone body with deionized water for approximately 3 hr;
(x) cryopreserving the decellularized cancellous bone block;
(y) lyophilizing the decellularized cancellous bone body;
(z) sterilizing the decellularized cancellous bone body utilizing gamma radiation.

In one aspect of the present disclosure, the cancellous bone body is a cancellous femoral head.

In one aspect of the present disclosure, the cancellous bone body is fragmented in blocks.

In one aspect of the present disclosure, the cancellous bone body is washed with a high-pressure water-jet spray that has a pressure equal or higher to 160 psi.

In a preferred aspect of the present disclosure, the cancellous bone body is immersed in a mixture of alcohol-chloroform at a 1:1 ratio for approximately 24 hr with gentle shaking at the speed of approximately 50 rpm at room temperature.

In a preferred aspect of the present disclosure, the cancellous bone body is placed in distilled water under continuous rotation at approximately 80 rpm for approximately 24 hr.

In a preferred aspect of the present disclosure, the cancellous bone body is deproteinized in 4% sodium hypochlorite for approximately 24 hr.

In a preferred aspect of the present disclosure, processing solution is a buffer.

In a most preferred aspect of the present disclosure, the buffer is phosphate-buffered saline.

In a preferred aspect, the non-ionic detergent is 1% Triton X-100.

In a preferred aspect of the present disclosure, the DNase and RNase are replaced every 12 hr with freshly prepared DNase and RNase.

In a preferred aspect of the present disclosure, the cancellous bone body is immersed in phosphate-buffered saline at 37° C. under continuous shaking when treated with DNase and RNase.

In one aspect of the present disclosure, the decellularized cancellous bone body contains less than approximately 50 ng dsDNA per mg ECM dryweight.

In a preferred aspect of the present disclosure, the decellularized cancellous bone body contains less than approximately 10 ng dsDNA per mg ECM dryweight.

In one aspect of the present disclosure, the decellularized cancellous bone body contains less than 200 bp DNA long fragments.

In one aspect of the present disclosure, the decellularized cancellous bone body does not have visible nuclear material by Hematoxylin and Eosin staining.

In a preferred aspect of the present disclosure, the cryopreservation method utilized is freeze-drying.

In one aspect of the present disclosure, the level of endotoxin in the decellularized cancellous bone body is less than 0.05 endotoxin units per mL.

In one aspect of the present disclosure, the level of endotoxin in the decellularized cancellous bone body is less than 0.02 endotoxin units per mL.

In one aspect of the present disclosure, the high-pressure water-jet spray enables the destruction of cell membranes and the removal of debris and facilitates the alcohol-chloroform treatment to gain access to the deeper parts of the bone for defatting.

In one aspect of the present disclosure, defatted bovine bone bodies are demineralized by immersing the bovine bone bodies in 0.6M HCl for 24 h with gentle rotation.

In another embodiment, there is provided a new decellularization process for bovine bone scaffolds (DCC) and compared its efficacy to the routine demineralized process (DMB) in-vitro. The initial common preparation process for both scaffolds began with a high hydrostatic pressure wash which enabled destruction of cell membranes, and removal of debris to facilitate subsequent alcohol-chloroform treatment to gain access to the deeper parts of the bone for defatting. Both groups of bone scaffolds exhibited a clean yellowish-white porous sponge-like appearance. The DCC scaffolds grossly showed more polished, cleaner, and whiter background compared to DMB scaffolds. Without being bound to any particular theory, the present invention proposes that the differences are possibly attributed to the more effective chemical effect of the NaOCL bleaching agent.

In certain embodiments, the chemical processing of the cancellous bovine bone scaffold or body takes two weeks followed by another seven days of enzymatic process prior to freezedrying.

In certain embodiments, the decellularization process of DCC of the present disclosure completely removes cells and nuclei from osteocyte lacunae.

In some embodiments, the decellularization process of DCC of the present disclosure removes cells and nuclei from osteocyte lacunae in a more efficient manner than the demineralization process of DMB.

In certain embodiments, total lipid reduction assessed by Oil Red O staining is at least 50%.

In a preferred embodiment, total lipid reduction assessed by Oil Red O staining is at least 60%.

In a most preferred embodiment, total lipid reduction assessed by Oil Red O staining is at least 70%.

In certain embodiments, the bovine bone scaffolds of the present disclosure are suitable for multiple surgical fields, including dental surgery, neurosurgery, and orthopedic surgery.

In preferred embodiments, the residual level of nucleic acid after DNase and RNase treatment content meets recommended levels for safe transplantation of biological tissue scaffolds.

In some embodiments, the bovine bone scaffolds of the present disclosure are suitable may be used as bone fillers and as structural support in moderate load-bearing areas in clinical applications.

In some embodiments, the decellularized cancellous bone body is sterilized with gamma radiation at 25 kGy.

In preferred embodiments, DCC scaffolds support attachment and growth of human osteoblast cells.

### EXPERIMENTAL EXAMPLES

The disclosure will be more fully understood upon consideration of the following non-limiting Examples. It should be understood that these Examples, while indicating preferred embodiments of the subject technology, are given by way of illustration only. From the above discussion and these Examples, one skilled in the art can ascertain the essential characteristics of the subject technology, and without departing from the spirit and scope thereof, can make various changes and modifications of the subject technology to adapt it to various uses and conditions.

As is evident from the foregoing description, certain aspects of the present disclosure are not limited by the particular details of the examples illustrated herein, and it is therefore contemplated that other modifications and applications, or equivalents thereof, will occur to those skilled in the art. It is accordingly intended that the claims shall cover all such modifications and applications that do not depart from the spirit and scope of the present disclosure.

Moreover, unless defined otherwise, all technical and scientific terms used herein have the same meaning as commonly understood by one of ordinary skill in the art to which the disclosure belongs. Although any methods and materials similar to or equivalent to or those described herein can be used in the practice or testing of the present disclosure, the preferred methods and materials are described above.

### Example 1

### Gross Morphology of Processed Bone Scaffolds

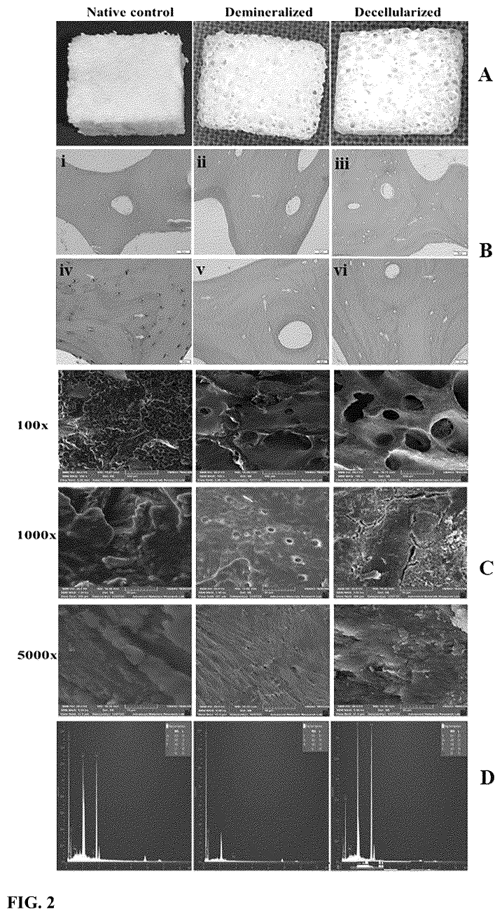

The gross morphology of the processed DMB and DCC scaffolds were analyzed to examine the impact of the physical, chemical and enzymatic treatment. Both types of scaffolds appeared white and clean, without any soft tissue attachments or debris within the pores. Effective removal of fat and red bone marrow was observed in both DMB and DCC scaffolds exhibiting a well porous sponge-like morphology (FIG. 2A).

### Histological Analysis

Histological examination of lyophilized DMB scaffolds showed an intact general structure of cancellous bone trabeculae with preserved lacunae and some containing osteocytes. At the same time, the lyophilized DCC scaffolds also presented with an intact general cancellous structure with well-preserved empty lacunae, demonstrating numerous amounts of missing osteocytes. Both scaffolds exhibited contrast histological features compared to the intact native bone matrix that demonstrated numerous amount of osteocytes with well-preserved cell morphology (FIG. 2B i and iv) in the lacunae. The naturally porous structure of the extracellular bone matrix was well maintained in both the DMB and DCC scaffolds. While the cellular components of the DMB bone showed deterioration of cellular components with the visible appearance of remnant pyknotic nuclei (FIG. 2B ii and v), the number of empty lacunae without traces of nuclei were comparatively more numerous in DCC scaffolds (FIG. 2B iii and v). Osteoblasts and osteoclast around the periphery of the bones were not seen in both demineralized and decellularized bone scaffolds. Fat cells were also absent in both scaffolds.

### SEM-EDS Evaluation of Scaffold

Morphological assessment at the ultrastructural level of both DMB and DCC scaffolds using SEM revealed the clean surface and pore architecture (FIG. 2C) exhibiting explicit natural porous microstructure and three-dimensional interconnectivity than the native control bone samples. High magnification at 1000× and 5000× revealed the clean and bare surface morphology in the treatment groups compared to native bones, where the deeper bone structures were not visible and covered completely by a layer of fat and debris. The protein phase (collagen matrix) was observed in the DMB scaffold, while clusters of calcified filaments (mineral phase) are visible in the deproteinized and decellularized SEM micrograph. The untreated control native bone (mineral and protein phases together) showed a compact surface morphology.

EDS's surface analysis chemical composition (Oxford Instruments X-max 50 EDS, UK) detected sodium, calcium, phosphorus, and oxygen in the DMB and DCC scaffolds (FIG. 2D). The quantitative chemical analysis obtained is shown in Table 1. There was a significant reduction in the amount of calcium in both DMB (0.1±0.0; p<0.0001) and DCC (29.0±0.3; p<0.001) scaffolds when compared to the native control bone samples (41.8±0.8). However, the percentage of calcium in the DMB group was significantly lower than that of the DCC group (0.1±0.0 vs. 29.0±0.3; p<0.0001; Table 1). The amount of phosphorous in DCC scaffolds (14.8±0.2) was closer to native bone (16.6±0.5), while it was undetectable in DMB scaffolds.

### Table. 1—Percentage of Surface Chemical Elements Detected in DMB and DCC Scaffolds Compared to Native Control.

TABLE 1

| | Element composition (Wt %) | | | |
|---|---|---|---|---|
| Scaffold type | Ca | P | Na | O |
| Native Control | 41.8 ± 0.8 | 16.6 ± 0.5 | 1.2 ± 0.3 | 39.8 ± 1.0 |
| DMB | 0.1 ± 0.0 * | — | — | 28.3 ± 0.4 |
| DCC | 29.0 ± 0.3** | 14.8 ± 0.2 | 0.5 ± 0.1 | 41.5 ± 0.5 |

DMB—demineralized bovine cancellous bone; DCC—decellularized bovine cancellous bone (* p < 0.001; **p < 0.0001)

When the porosity between DMB and DCC scaffolds were compared, the DCC scaffolds generally showed higher porosity (FIG. 3a, 3b). Under 100× magnification, DCC scaffolds had a relatively larger pore size (365.87±62.2 μm; p<0.05; FIG. 3c), showing deeper porosity with obvious interconnectivity compared with DMB scaffold micro-architecture (202.92±102.11 μm). The porosity depth in the DMB scaffold was generally not very clearly seen. No feature of microfractures was observed at 5000× magnification on the surface and deeper pores of both DMB and DCC scaffolds.

### Lipid Content

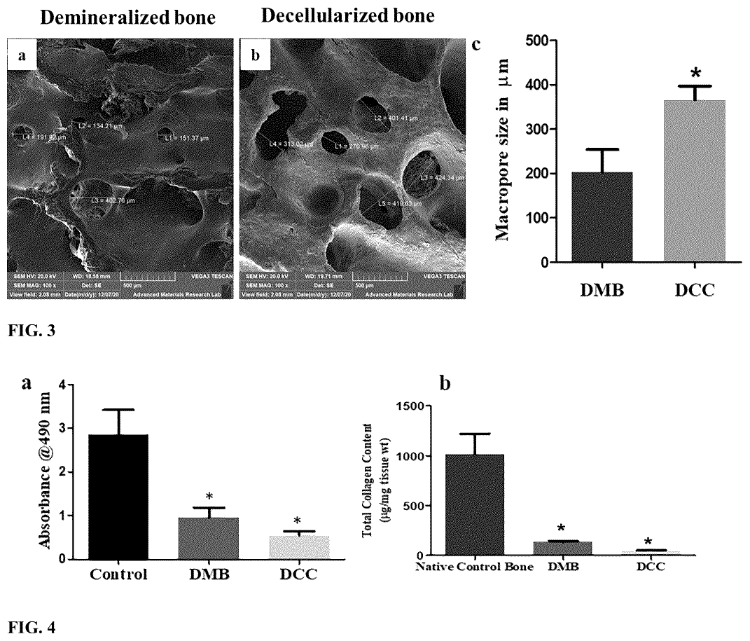

Oil red O staining confirmed the efficient removal of residual lipid content in DMB and DCC scaffolds. Oil Red O combines with triacylglycerol to give out jacinth lipid droplets, which can be spectrophotometrically quantified. Compared to native control bone, DMB and DCC scaffolds exhibited a significant reduction in total lipid content (p<0.05; FIG. 4a).

### Collagen Content

Total collagen assay revealed a significant reduction in the total collagen content of both DMB (131.22±10.79 μg/mg ECM wt; p<0.05) and DCC (45.45±4.69 μg/mg ECM wt; p<0.05; FIG. 4b) scaffolds as compared to the native control bone (1010.73±170.42 μg/mg ECM wt). The data further confirmed the removal of a significantly larger amount of collagen following DCC treatment compared to DMB treatment.

### FTIR Analysis of Bone Samples

The FTIR spectra recorded in the DMB and DCC scaffolds showed significant changes compared to the native control bone, and specific peaks of interest have been marked in the spectrum (FIG. 5). A broad absorption peak of cholesterol is evident a5 3412 cm-1 in native control bone but was not found in both DMB and DCC bone scaffolds suggesting the effective removal of fat from the bone samples by both treatment processes (FIG. 5a). Other regions of interest including collagen that is depicted as amide I (1585 1720 cm-1), amide II (1500-1600-1), amide III (1250 1350 cm-1); and carbonate (850 890 cm-1) and phosphate (900 1200 cm-1) are also demonstrated (FIG. 5b). Amide I results from peptide bond C═O stretch, amide II from mixed C N stretch and N H in-plane bend, and amide III results from mixed C N stretch and N H in-plane bend.

### Quantification of Residual Nucleic Acid Content in DCC Bone Samples

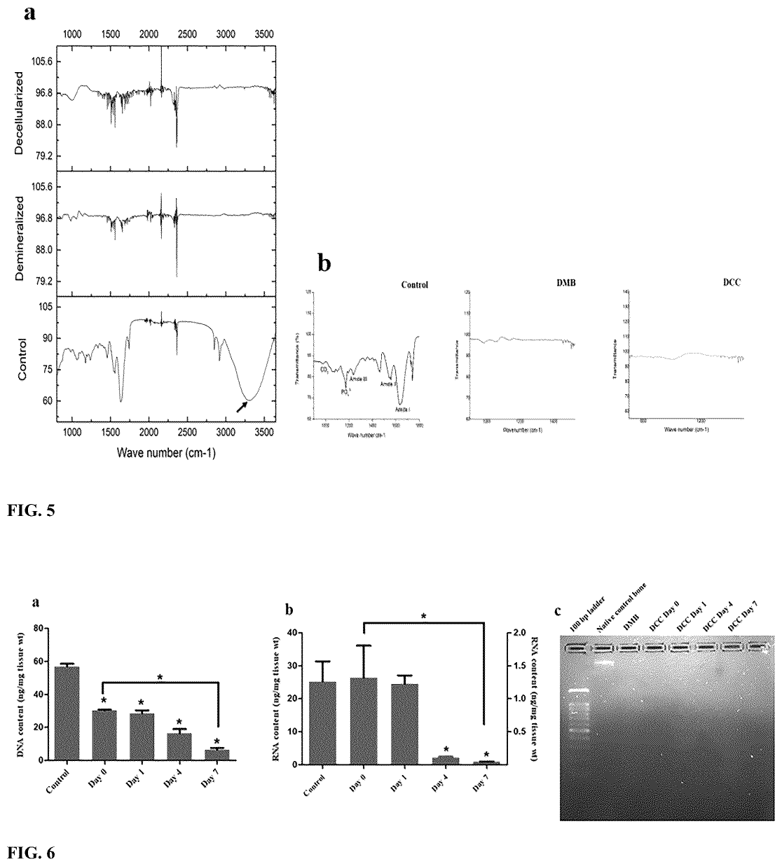

To evaluate the efficacy of DNase treatment, both scaffolds' total DNA and RNA content were extracted, quantified and compared. Native bovine bone served as the control in the experiment. The quantification of nucleic acids in both native cancellous bone blocks and DCC scaffold is shown in FIG. 6a and FIG. 6b. Total residual DNA/RNA obtained was normalized by the dry weight of each bone sample and interpreted.

The average total DNA content obtained was 56.9±2.37 ng/mg dry wt of ECM for native control bone and 30.4±0.49 ng/mg dry wt of ECM (p 0.0001) in DCC scaffold. The DNA content in the DCC scaffold was significantly reduced to 28.5±2.69 ng/mg dry wt of ECM (p<0.0001) on day 1, and further declined to 16.33±3.68 ng/mg dry ECM wt (p 0.001) on day 4 and reduced to a low amount of 6.53±1.57 (p 0.0001) on day 7 of DNase treatment (Table 2).

The average total RNA content was 27.87±11.05 ng/mg dry wt of ECM for native control bone and 28.04±15.56 ng/mg dry wt of ECM for DCC scaffold. The RNA content in the DCC scaffold was reduced to 24.3±4.7 ng/mg dry wt of ECM on day 1, and further declined significantly to 0.1±0.03 ng/mg dry wt of ECM on day 4 (p<0.01) and finally reduced to a meager amount of 0.05±0.03 ng/mg dry wt of ECM (p<0.01) on day 7 of RNase treatment. There was a significant reduction in total RNA content from Day 0 to Day 7 of RNase treatment (Table 2; p<0.01). Agarose gel electrophoresis image revealed the presence of a visible DNA band of high molecular weight in the native control bone sample, whereas DMB and DCC scaffolds did not show the presence of any DNA bands (FIG. 6c).

Table 2. Nucleic Acid Quantification. Quantification of the Residual DNA/RNA Content in DCC Scaffolds Over One Week of DNase/RNase Treatment Compared to Native Control Bone.

| | DNA Concentration | RNA Concentration |
|---|---|---|
| | (ng/mg dry wt | (ng/mg dry wt |
| | of ECM ± SEM) | of ECM ± SEM) |
| Control | 56.9 ± 2.37 | 27.87 ± 11.05 |
| Day 0 | 30.4 ± 0.49 | 28.04 ± 15.56 |
| Day 1 | 28.5 ± 2.69 | 24.30 ± 4.70 |
| Day 4 | 16.3 ± 3.68 | 0.10 ± 0.03 |
| Day 7 | 6.53 ± 1.57 | 0.05 ± 0.00 |

*p < 0.0001;
**p < 0.01

### Example 2

### Biochemical Analysis

### Compression Test

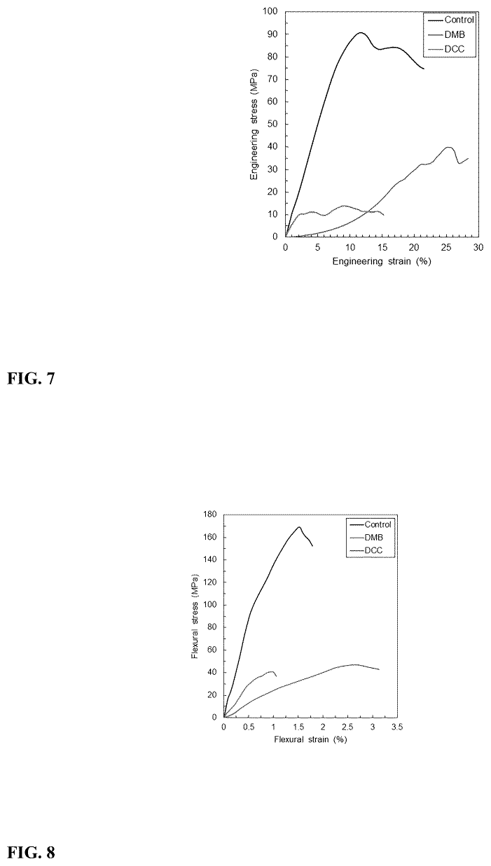

Compressive engineering stress and engineering strain (SS) curves of all sample groups are shown in FIG. 7. All samples exhibited a typical SS behavior of bone [11]: elastic and plastic regions except the DMB 3 scaffold. In the elastic region, the stress increases linearly with increasing strain, then a point in the linear relationship finally ends when the bone begins to yield. The yield point marks the beginning of plastic deformation, and then the plastic deformation occurs upon compressive test afterward. Since there is no significant boundary between elastic and plastic regions, 0.2% strain offset [12], i.e., 0.2% proof stress, was applied as the yield point in this study. The 0.2% proof stresses and the ultimate compressive stresses of control samples were higher than other scaffolds, while the DCC showed the lowest 0.2% and ultimate compressive stresses.

### Three-Point Bending Test

The relation between flexural stress and strain of all the sample groups is shown in FIG. 8. The flexural SS curves also exhibit elastic and plastic deformation behavior of bones and are similar to the SS curve of the compressive test. The maximum flexural stress of the DCC scaffolds was higher than the DMB, while the DMB had a higher total strain compared to the DCC. The average flexural elasticities of the native control, DMB, and DCC scaffolds are 17.2 GPa, 1.7 GPa, and 5.9 GPa, respectively.

### Endotoxin Testing

The level of endotoxin present in both DMB and DCC scaffolds were observed to be 0.010025 and 0.015058 EU/ml respectively which is much below the FDA limit of 0.05 endotoxin units/ml [13]. The demineralization and decellularization processes successfully reduce endotoxin contamination and concentration in both DMB and DCC scaffolds.

### In Vitro Cell Viability

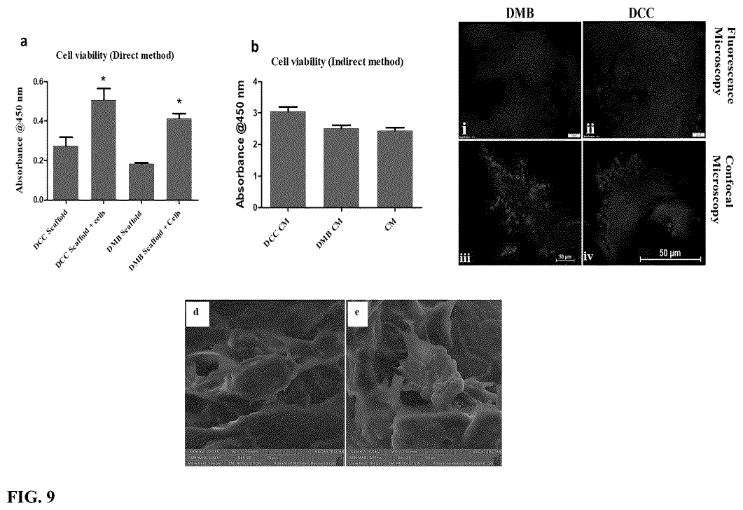

Cell viability study revealed that both DMB and DCC scaffolds supported growth and proliferation of osteoblast cells. The present study addressed both the direct and indirect methods for evaluating biocompatibility of the scaffolds. In the direct method, cell proliferation was found to be significantly high in both DMB and DCC scaffolds in comparison to the control (FIG. 9a; p 0.05). Although, cell proliferation was observed to be higher in DCC scaffolds in comparison to cell seeded DMB scaffolds, the difference was not significant suggesting an equal efficiency for both the scaffolds in supporting osteoblast proliferation. In indirect method, cell proliferation in the presence of DMB and DCC scaffold extract was found to be comparable with the complete control medium suggesting that there is no inhibition in growth and proliferation of cells upon exposure to both scaffold extracts (FIG. 9b). The data suggests that the porous nature of the scaffolds provided ambient conditions for adhesion, colonization and proliferation of osteoblast cells, and enable adequate nutrient supply. It further imply that the processing techniques were able to remove residual cytotoxic chemicals at each processing step.

Cell Seeding onto Scaffolds and DAPI Staining

DAPI staining of the cell seeded scaffolds revealed that both DMB and DCC scaffolds are conducive substrates for cell attachment and spread. Osteoblast cell attachment were observed in sufficient densities in both DMB and DCC scaffolds (FIG. 9c).

### SEM Imaging of the Cell Seeded Scaffolds

SEM imaging showed osteoblast capability of cell attachment and interaction with the extracellular matrix in the DMB and DCC scaffolds. After seven days of culture, osteoblasts seeded on DMB scaffolds showed a healthy appearance with filopodia extending to the ECM (FIG. 9d). Similar cytoplasmic processes and osteoblast-substrate engagement were clearly visible in DCC scaffolds as well (FIG. 9e). Presence of intact collagen fibrils within the porous microstructure offer cell attachment sites that favor establishment of cell-matrix and cell-cell interactions through their extended filopodia.

### Mineralization Study

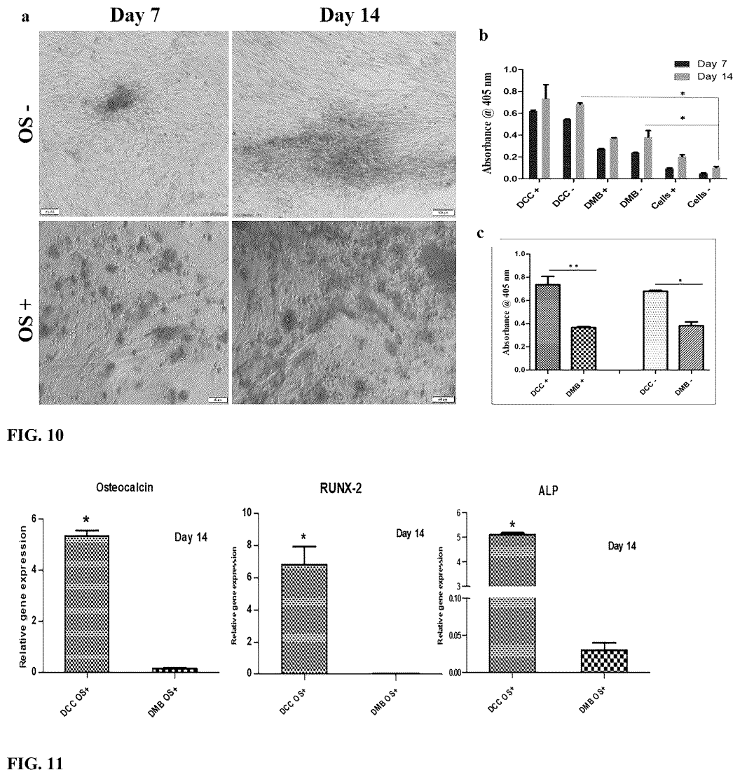

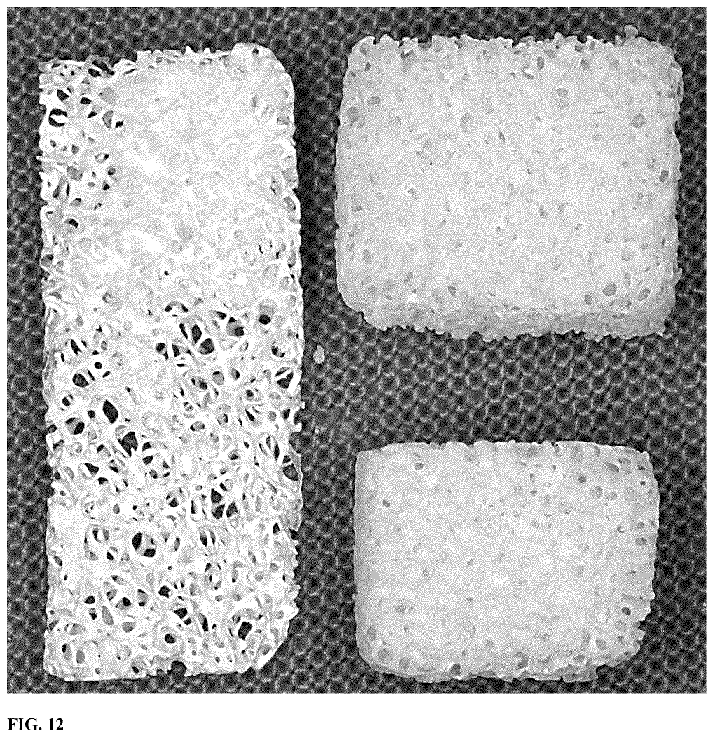

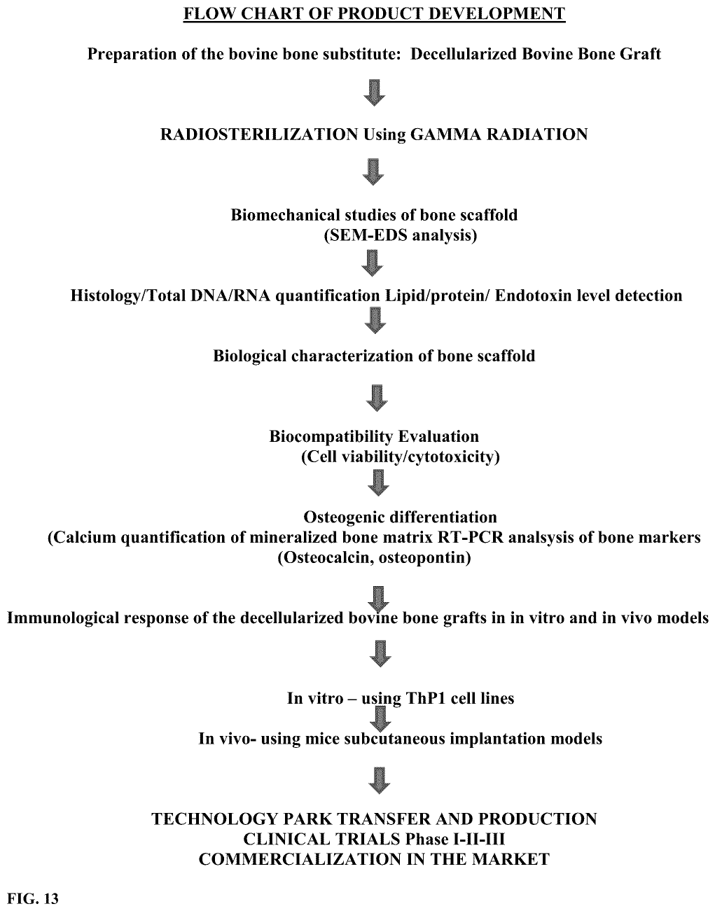

The effect of scaffolds on inducing osteoblast mineralization were studied in the presence and absence of osteogenic media at two specific time points, on day 7 and 14. Cells alone grown in the presence and absence of osteogenic media served as control in the experiment. Alizarin Red S staining experiments revealed significantly higher staining and nodule formation in cells supplemented with osteogenic media than cells without any supplements. Also, more evident staining was observed by day 14 of cell culture than day 7 (FIG. 10a).

When the osteoblast cells seeded DMB and DCC scaffolds were studied for their mineralization potential, it was observed that DCC scaffolds favored enhanced differentiation of osteoblasts than DMB scaffolds in the absence of osteogenic supplements. Quantification of Alizarin Red stain revealed that when compared to control cells grown for 14 days without supplements, mineralization was more evident in cells seeded on DMB (p<0.0001) and DCC scaffolds (p<0.0001) even in the absence of osteogenic supplements (FIG. 10b). When both the scaffolds were compared, mineralization was more rampant in cells seeded on DCC scaffolds than cells grown on DMB scaffold (p<0.001; FIG. 10c) irrespective of the presence or absence of osteogenic supplements.

### Gene Expression of Osteogenic Markers

Expression of the osteogenic markers such as alkaline phosphatase (ALP), osteocalcin (OCN), and RUNX2 in osteoblasts cultured on the DMB and DCC scaffolds for 14 days were analyzed by quantitative real-time PCR. In support to the findings from alizarin staining experiments, expression of the osteogenic markers were significantly higher in cells grown on DCC scaffolds than cells grown on DMB scaffolds for all the three osteogenic markers (p<0.0001; FIG. 11). It was also observed that the expression for the early osteogenic marker, RUNX2, was higher compared to ALP and OCN markers.

### Materials and Methods

### Preparation of Bovine Scaffolds

### Procurement of Bovine Bone

Fresh femur bones from 24-month-old calves were obtained from a local slaughterhouse. The whole femur was then transported to the laboratory immediately and stored at −80° C. until further use. Briefly, muscular soft tissue attachments around the femur were removed using a periosteal elevator. The femur head was separated from the shaft using a bone saw (JG210 Bone Cutting machine; Shandong China), and the long bone shaft was discarded. The outermost cortical layer of the bone surrounding the femoral head was removed, leaving behind the cancellous part. The cancellous femoral head was then shaped in three bone block forms.

### Processing of Bovine Cancellous Bone Scaffolds

The cancellous bone blocks were cleaned thoroughly in distilled water using a high-pressure water-jet spray with pressure not exceeding 160 psi. The water jet (Electric pump 100 W 12V water cleaner with pressure) flushed through its cancellous pores to physically remove all blood cells, fat, and other soft tissue debris. Delipidation was next performed by immersing the bone blocks in a mixture of alcohol-chloroform (1:1) for 24 h with a gentle shaking speed of 50 rpm at room temperature. The bovine bone blocks were then immersed in deionized water and rinsed thoroughly 3 times with 10 min intervals to remove the residual alcohol and chloroform. The bone blocks were then transferred to fresh distilled water and maintained in continuous rotation at 80 rpm (Shaker, SK-02, Zenith Lab co, China) for 24 h to ensure complete removal of alcohol and debris trapped in the deep pores. Overnight washing in mild rotation produced clean bone samples without any visible red blood cell contamination or the presence of fat. The washed and defatted bone blocks were then randomly divided into two groups.

The first group was processed to produce a lyophilized demineralized bovine cancellous bone (DMB) scaffold. The second group was treated to produce a lyophilized decellularized bovine cancellous bone (DCC) scaffold. A third group comprised of non-processed native bovine cancellous bone blocks act as a control. In Group I, the defatted bovine bone blocks was subsequently demineralized by immersion in 0.6 M HCL for 24 h with gentle rotation. At the end of acid treatment, these “demineralized bovine cancellous bone” (DMB) scaffolds were rinsed with sterile deionized water 3 times with 10 min intervals to ensure maximum removal of the residual acid. All these steps were performed under continuous shaking at room temperature in a rotatory shaker. In Group II, the defatted bovine bone blocks were decellularized, beginning with partial deproteinization by treating the bone in 4% sodium hypochlorite (NaOCL) for 24 h with gentle shaking at room temperature. The bone blocks were then washed thoroughly with deionized water for 72 h to eliminate residual chemicals. This was followed by rinsing in phosphatebuffered saline (PBS, Sigma Aldrich, USA) and then subjected to sequential washes in 0.01, 0.1, and 1% Sodium dodecyl sulfate (SDS) for 72 h. The SDS-treated bone scaffolds were next exposed to 1% Triton X-100 (Sigma Aldrich, USA) in deionized water to remove the residual SDS and cellular debris. At the end of the procedure, the treated scaffolds were subjected to enzymatic treatment. They were immersed in a solution of DNase (0.2 mg/ml; Sigma, USA) and RNase (1 μg/ml; Sigma, USA) in PBS at 370 C with continuous shaking using a magnetic shaker (Labnet, USA). The enzyme mixture was replaced every 12 h with freshly prepared DNase/RNase, and treatment was continued for 1 week. Samples were retrieved on days 0, 1, 4, and 7 for residual nucleic acid quantification. At the end of the enzymatic treatment, these “decellularized bovine cancellous bone” (DCC) scaffolds were rinsed thoroughly with sterile deionized water for 3 h with intermittent changes of water.

### Lyophilization and Gamma Sterilization

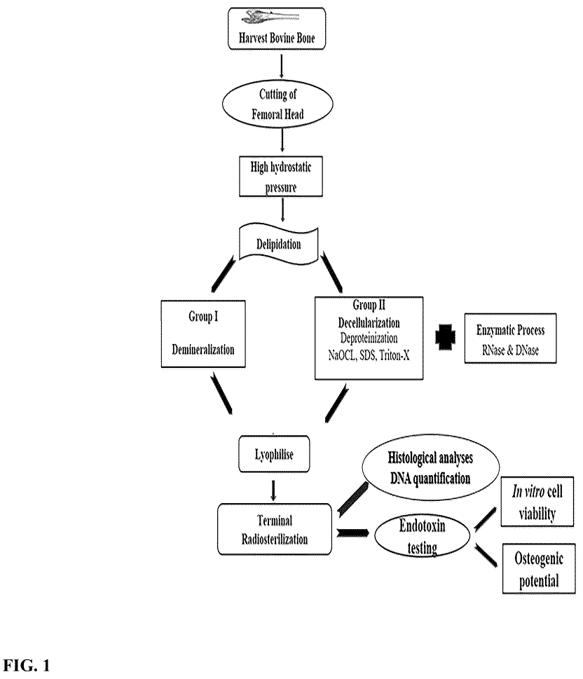

Both Group I DMB scaffolds and Group II DCC scaffolds were deep-freezed at −80 C for 4 h and lyophilized using Vir Tis BenchTop Pro with Omnitronics™ (SP Scientific, PA, USA). The temperature of the lyophilizer was set at −40° ° C., 7×10−2 milliBar pressure and the entire procedure lasted for 24 h. Following lyophilization, the DMB scaffolds and DCC scaffolds were packed separately in double-layer plastic pouches and were radio-sterilized using gamma radiation at 25 kGy. The main sequential processing steps for producing DMB and DCC scaffolds in this study are shown in FIG. 1.

### Physicochemical Characterization of Bovine Scaffolds

### Histological Analysis

Samples from radio-sterilized DMB scaffolds and DCC scaffolds and control native bovine cancellous bone blocks were fixed in 10% neutral buffered formalin (Thermo Fischer Scientific, USA), and decalcified using ready-made decalcificationsolution (Shandon™ TBD 1™ decalcifier, Thermo Fisher Scientific, USA) with periodic examination for softening of the samples. Upon completing the decalcification process, the bones were rinsed in running tap water thoroughly for 2 h followed by paraffin embedding and sectioning into 7 μm thick sections. Bone sections were stained with nuclear dye Hematoxylin and the counterstain Eosin (Sigma-Aldrich). Images of tissue sections were captured using an inverted microscope (Olympus IX73, Japan).

Scanning Electron Microscopy with Energy-Dispersive X-Ray Spectroscopy (SEM-EDS)

Surface topography and surface elemental analysis was performed using SEM-EDS. The DMB and DCC scaffolds, along with the native controls shaped in 5×2×2 mm sizes, were coated with gold using Quorum technologies SC7620 before SEM imaging. Tescan VEGA 3 XMU was used in the study. Elements, from three random regions across the scaffold samples, were detected by using Oxford Instruments X-max 50 EDS detector.

### Evaluation of Lipid Content

The residual lipid content present in the lyophilized DMB and DCC scaffolds was estimated by Oil Red O quantification. About 100 mg of lyophilized gamma sterilized DMB and DCC scaffolds were incubated in 0.5 mL of Oil Red O solution (Sigma, USA) for 15 min at room temperature. Native control bone blocks served as control. The scaffolds and native bone blocks were washed in deionized water four times, followed by extraction of the oil red stain with 0.25 mL 100% isopropanol, and the absorbance was measured at 490 nm using a multilabel plate reader (Multiskan™ GO, Thermo Scientific, USA).

### Quantification of Total Collagen

The post-treatment collagen content in the DMB and DCC scaffolds was quantified using the Total Collagen Assay Kit (Abcam, USA), following the manufacturer's instructions. The assay is based on alkaline hydrolysis of the bone samples releasing free hydroxyproline, which is later oxidized and quantified at OD 560 nm. All procedures were carried out in triplicates.

### Fourier-Transform Infrared Spectroscopy (FTIR)

The FTIR-ATR spectra of the DMB and DCC scaffolds were collected using Jasco FT/IR-6300 (Tokyo, Japan). Spectra were recorded with a resolution of 2 cm-1 with 20 scans over the range 4000-400 cm-1 at a constant temperature of 25° C. Three replicates of each sample were developed. Data analysis was performed using Origin Pro 8.5 software, and the presence of the ECM organic components, including collagen, fat, and carbonate, were evaluated. Spectra analysis was conducted in the attenuated total reflection (ATR) sampling mode and baseline corrected.

### Quantification of Residual Nucleic Acid in DMB and DCC Scaffolds

In Group II, the residual DNA/RNA content in lyophilized DCC scaffolds was determined using a DN/RN easy Blood and Tissue kit (Qiagen, USA). The lyophilized DCC scaffolds (n=5) were pulverized, and 100 mg of scaffold powder was used for DNA/RNA extraction. Total dsDNA/RNA was extracted following the manufacturer's protocol with some modifications at days 0, 1, 4, and 7 of DN/RNase treatment, respectively. The quality and concentration of the extracted total DNA and RNA were measured using the NanoDrop ND1000 (Thermo Scientific, Waltham, MA, USA) and expressed as nanograms per milligram of ECM dry weight. The extracted DNA was further loaded in 1% agarose gel, run along a100 bp DNA ladder to check the residual DNA fragment length. In Group I lyophilized DMB scaffolds, only total DNA content was estimated as described above. Samples from Native bovine bone block served as the control.

### Biomechanical Characterization of Bovine Scaffolds

### Compression Test

Lyophilized gamma-irradiated DMB and DCC scaffolds were subjected to Quasi-static compressive tests in an air-dry condition. A universal testing machine, Model 5 ST (Tinius Olsen Single Column Testing Systems, USA), equipped with a 5 kN load cell, was used for the analysis. Specimens were tested at a 1 mm/min crosshead speed. All specimens were loaded until compressive failure. Native control bone samples served as control.

### Three-Point Bending Test

Lyophilized, gamma-irradiated DMB and DCC scaffolds shaped into 25 mm×5 mm×2 mm sizes were prepared for a three-point bending test. A total of 6 bone samples in each group were tested using three-point bending by a universal testing machine, Model 5 ST (Tinius Olsen Single Column Testing Systems, USA), at a 1-mm/min crosshead speed. The failure point of each specimen was determined by analysis of the flexural stress-strain curves. Native control bone samples served as control.

### Biological Evaluation of Bovine

### Scaffold Human Osteoblast Culture

Human osteoblast cells (HOB) used for biocompatibility evaluation were obtained from Addexbio (Cat no: P0004010; USA). The cells were cultured in a complete medium containing Dulbecco's modified Eagle's medium-Ham F12 (DMEM F12, Gibco®, USA) supplemented with 10% fetal bovine serum (FBS) and 1% antibiotics (Sigma, USA) in a humidified atmosphere at 37° C. and 5% CO2. The medium was replaced every three days and cells were passaged on approaching 80 90% confluence, using 0.25% trypsin-EDTA solution (Sigma, USA). HOB cells harvested in passages 3-6 were used for the experiments in the study.

### Endotoxin Testing

Before the initiation of the in vitro experiments, the DMB and DCC scaffolds were tested for endotoxin contamination using the chromogenic LAL (Limulus amebocyte lysate) assay (Lonza, Basel, Switzerland). The assay was performed according to the manufacturer's instructions.

For the preparation of the samples, gamma-sterilized DMB and DCC scaffolds were incubated in endotoxin-free water (1 mL) for 24 h at 37 C to extract any potential endotoxin release from the bone scaffolds. After incubation time, 50 μL of the supernatant was employed in the LAL test. A standard curve was prepared using the positive controls provided by the manufacturer within the LAL kit and pure endotoxin-free water was used as the negative control.

### In Vitro Cell Viability

The biocompatibility of DMB and DCC scaffolds was evaluated by both direct and indirect methods and the amount of metabolically active cells was quantified by XTT assay (Roche Diagnostics, Mannheim, Germany). For the direct method, cells were seeded directly on the scaffolds at a density of 5×10⁴ cells/scaffold and incubated for a period of 24-48 h. Before viability testing, the fixation rings were removed, and the scaffolds were transferred to new 12-well multiwell plates. The scaffolds were then incubated with XTT reagent for 4 h at 37° C. 100 mL replicates of the supernatant were then transferred into a 96-well plate, and absorbance was measured at 450 nm. For the indirect method, HOB cells at a density of 3×103 cells were seeded on 96 well culture plates and incubated overnight for cell adherence. On the same day, scaffolds along with fixation rings were incubated with complete medium overnight. The medium was aspirated out from the osteoblasts cells and replaced with the scaffold extracts and incubated further for 48 h. XTT assay was performed in order to assess the effect of scaffold extracts on cell proliferation/viability. In both the methods, culture medium with scaffolds holding the glass rings served as the negative control.

### Cell Seeding on Scaffolds

DMB and DCC scaffolds were placed into 12-well multiwell plates (Sigma Aldrich, USA). The scaffolds were weighed down with sterile autoclaved stainless rings in order to keep them in position and prevent from floating in the cell culture medium. Before seeding the cells, the scaffolds were incubated with complete culture medium overnight in a CO₂ incubator. The medium was then aspirated out and the scaffolds were loaded slowly with cells in the center at a concentration of 5.0×10⁴ cells per scaffold in a total volume of 50-75 mL. The multiwell plates were then incubated carefully without movement for initial cell attachment for 2-3 h. The final culture volume was then made up to 1-2 ml and the cell seeded scaffolds were further incubated for 2-3 days for cell attachment.

### DAPI Staining

The stainless steel rings were carefully removed from the cell seeded DMB and DCC scaffolds and the scaffolds were placed onto new culture wells. The scaffolds were then carefully washed three times with sterile PBS and stained with a mounting medium with DAPI (Abcam, USA) for 5-10 mins and observed under fluorescence microscope (Olympus, Japan) for cell attachment in the first 24 hrs. Images were captured using both florescence and confocal microscopes (Nikon Eclipse Ti-S, Nikon Instruments Inc., USA).

### Scanning Electron Microscopy (SEM) of Cell Seeded Scaffolds

Osteoblast cell seeded DMB and DCC scaffolds were subjected to SEM imaging. Briefly, cells were seeded on scaffolds and maintained in complete DMEM-F12 medium for 7 days. The cell seeded scaffolds were then fixed in 2.5% glutaraldehyde for 1 h, followed by progressive dehydration in ethanol and coated with gold using Quorum technologies SC7620 prior to SEM imaging. Tescan VEGA 3 XMU was used in the study.

### Mineralization Study

HOB cells seeded onto DMB and DCC scaffolds were evaluated for their mineralization potential in order to assess their differentiation potential. Both DMB and DCC scaffolds were seeded with HOB cells at a density of 5×10⁴ cells/scaffold placed on 24-well plates, in the presence/absence of osteogenic factors, and matrix mineralization was evaluated at two time points (14 and 21 days). Calcium deposition by the osteoblast cells was determined by Alizarin Red S staining (ARS). The osteogenic induction factors used include 10-8M dexamethasone, 10 mM beta glycerophosphate and 50 μg/ml ascorbic acid. HOB cells alone seeded in the presence or absence of osteogenic factors served as control in the experiment. At specific time points, the cells and cell seeded scaffolds were washed in PBS and fixed in 4% paraformaldehyde solution for 15 min at room temperature. The cell/scaffold samples were stained with alizarin red s solution (40 mM) for 20 min at room temperature with gentle rocking. After removal of alizarin red s solution, the cell/scaffold samples were washed three times with deionized water and the stained cells and scaffolds were imaged using an inverted phase contrast tissue culture microscope (Olympus, CKX 41, NY, USA).

For calcium quantification, 10% acetic acid (v/v) was added to the Alizarin Red stained cells/scaffolds and incubated for 30 min at RT with agitation. Cells were scraped out from the culture wells, vortexed for 30 s, and incubated for 10 min at 85° C. Whereas the cell seeded scaffolds were directly vortexed and incubated at 85° C. Samples were centrifuged for 15 min at 12000 rpm and the cell lysates were then collected as supernatant and were then quantified using a plate reader at absorbance 405 nm. A standard calibration curve for alizarin dye was prepared for quantification of total calcium release.

### Gene Expression of Osteogenic Markers by Real Time RT PCR

HOB cells grown on the DMB and DCC scaffolds for 14 days were evaluated for the expression of osteogenic genes that include alkaline phosphatase (ALP), osteocalcin (OC) and Runt-related transcription factor 2 (RUNX-2). The total RNA was extracted using RNeasy kit (Invitrogen, USA) following manufacturer's instructions. The quality and concentration of the RNA samples were measured using the NanoDrop ND1000 (Thermo Scientific, Waltham, MA, USA). First strand cDNA was synthesized by reverse transcriptase using the Highscript cDNA syntheis kit (Thermoscientific, USA) and the expression of osteogenic markers was quantified using 5×FIREPOI SYBRGreen Mix (Solisbiodyne). The gene specific primer sequences were used for the reaction and glyceraldehyde-3-phosphate dehydrogenase (GAPDH) was used as an internal control for data normalization. HOB cells grown in the absence of osteogenic medium served as control. The qPCR amplification was conducted as follows: initial denaturation at 95° C. for 10 min, followed by 40 cycles at 95° C. for 30 s, 60° C. for 1 min, and 72° C. for 1 min. The reaction was performed using StepOne Thermocycler (Applied Biosystems, USA) and the results were quantified using the DDCt relative quantification method. To examine the differences in performance as a bone graft scaffold between DMB and DCC scaffold, we evaluated their efficacy in terms of removal of all cellular materials, including nucleic acids and lipids, porosity, preservation of proteins and minerals, mechanical strength, as well as their in vitro cytotoxicity, substrate support for osteoblast growth, osteogenic gene expression and mineralization potential of ECM.

### Data and Statistical Analysis

Statistical analysis was carried out using GraphPad Prism 5. Data were expressed as mean±SEM. One-way analysis of variance (ANOVA) with Bonferroni's post hoc test was applied to identify the differences between the study groups, and unpaired t-test was used whenever differences between two groups were studied. A p-value of p<0.05 was considered to be statistically significant.

As is evident from the foregoing description, certain aspects of the present disclosure are not limited by the particular details of the examples illustrated herein, and it is therefore contemplated that other modifications and applications, or equivalents thereof, will occur to those skilled in the art. It is accordingly intended that the claims shall cover all such modifications and applications that do not depart from the spirit and scope of the present disclosure.

Moreover, unless defined otherwise, all technical and scientific terms used herein have the same meaning as commonly understood by one of ordinary skill in the art to which the disclosure belongs. Although any methods and materials similar to or equivalent to or those described herein can be used in the practice or testing of the present disclosure, the preferred methods and materials are described above.

### REFERENCES

[1] Rodriguez A E, Nowzari H. The long-term risks and complications of bovine-derived xenografts: A case series. J Indian Soc Periodontol. 2019 September-October; 23(5):487-492. doi: 10.4103/jisp.jisp_656_18. PMID: 31543624; PMCID: PMC6737859.
[2] Fay Goldstep. Bone Grafts For Implant Dentistry: The Basics; 2015.
[3] Oryan A, Alidadi S, Moshiri A, Maffulli N. Bone regenerative medicine: classic options, novel strategies, and future directions. J Orthop Surg Res. 2014 Mar. 17; 9(1):18. doi: 10.1186/1749-799X-9-18. PMID: 24628910; PMCID: PMC3995444.
[4] You L, Weikang X, Lifeng Y, Changyan L, Yongliang L, Xiaohui W, Bin X. In vivo immunogenicity of bovine bone removed by a novel decellularization protocol based on supercritical carbon dioxide. Artif Cells Nanomed Biotechnol. 2018; 46(sup2):334-344. doi: 10.1080/21691401.2018.1457044. Epub 2018 May 4. PMID: 29726299.
[5] Gardin C, Ricci S, Ferroni L, Guazzo R, Sbricoli L, De Benedictis G, Finotti L, Isola M, Bressan E, Zavan B. Decellularization and Delipidation Protocols of Bovine Bone and Pericardium for Bone Grafting and Guided Bone Regeneration Procedures. PLOS One. 2015 Jul. 20; 10(7):e0132344. doi: 10.1371/journal.pone.0132344. PMID: 26191793; PMCID: PMC4507977.
[7] Gilpin A, Yang Y. Decellularization Strategies for Regenerative Medicine: From Processing Techniques to Applications. Biomed Res Int. 2017; 2017:9831534. doi: 10.1155/2017/9831534. Epub 2017 Apr. 30. PMID: 28540307; PMCID: PMC5429943.
[8] Hashimoto Y, Funamoto S, Kimura T, Nam K, Fujisato T, Kishida A. The effect of decellularized bone/bone marrow produced by high-hydrostatic pressurization on the osteogenic differentiation of mesenchymal stem cells. Biomaterials. 2011 October; 32(29):7060-7. doi: 10.1016/j.biomaterials.2011.06.008. Epub 2011 Jul. 2. PMID: 21724252.
[9] Fröhlich M, Grayson W L, Wan L Q, Marolt D, Drobnic M, Vunjak-Novakovic G. Tissue engineered bone grafts: biological requirements, tissue culture and clinical relevance. Curr Stem Cell Res Ther. 2008 December; 3(4):254-64. doi: 10.2174/157488808786733962. PMID: 19075755; PMCID: PMC2773298.
[10] Villa M M, Wang L, Rowe D W, Wei M. Effects of cell-attachment and extracellular matrix on bone formation in vivo in collagen-hydroxyapatite scaffolds. PLOS One. 2014 Oct. 16; 9(10):e109568. doi: 10.1371/journal.pone.0109568. PMID: 25329879; PMCID: PMC4199619.
[11] Xia S, Lan Q, Su S, Wang X, Xu W, Liu Z, et al. The role of furin cleavage site in SARS-COV-2 spike protein-mediated membrane fusion in the presence or absence of trypsin. Signal Transduction and Targeted Therapy. 2020; 5:92.
[12] Coutard B, Valle C, de Lamballerie X, Canard B, Seidah N G, Decroly E. The spike glycoprotein of the new coronavirus 2019-nCOV contains a furin-like cleavage site absent in CoV of the same clade. Antiviral Research. 2020; 176:104742.
[13] e Greef J C, Slutter B, Anderson M E, Hamlyn R, Landa R O C, McNutt E J, et al. Protective role for the N-terminal domain of α-dystroglycan in Influenza A virus proliferation. Proceedings of the National Academy of Sciences. 2019; 116:11396-401.

1. A process for the decellularization of a bovine bone scaffold, comprising the sequential steps of:
(a) washing a bovine cancellous bone body with a distilled pressurized water;
(b) immersing the cancellous bone body in a mixture of alcohol-chloroform at a 1:1 ratio for approximately 24 hr with gentle shaking at the speed of approximately 25 to 75 rpm at room temperature;
(c) rinsing the cancellous bone body in deionized water at least three times;
(d) placing the cancellous bone body in distilled water under rotation at approximately 50 to 100 rpm for a time range of approximately 12 to 36 hr;
(e) deproteinizing the cancellous bone body in approximately 4% sodium hypochlorite for a time range of approximately 18 to 30 hr at room temperature;
(f) rinsing the cancellous bone body in a processing solution;
(g) washing the cancellous bone body sequentially in 0.01, 0.1, and 1% sodium dodecyl sulfate for approximately 72 total hrs;
(h) treating the cancellous bone body with a non-ionic detergent;
(i) treating the cancellous bone body with DNase and RNase for approximately a week, wherein at the end of all treatment steps, the cancellous bone body is decellularized;
(j) rinsing the decellularized cancellous bone body with deionized water for approximately 3 hr;
(k) cryopreserving the decellularized cancellous bone body;
(l) lyophilizing the decellularized cancellous bone body; and
(m) sterilizing the decellularized cancellous bone body utilizing gamma radiation.

2. The process of claim 1, wherein the cancellous bone body is a cancellous femoral head.

3. The process of claim 1, wherein the cancellous bone body is fragmented in blocks.

4. The process of claim 1, wherein the cancellous bone body is washed as in step (a) with a high-pressure water-jet spray that has a pressure not exceeding 160 psi.

5. The process of claim 1, wherein the gentle shaking is set at the speed of approximately 50 rpm at room temperature.

6. The process of claim 1, wherein the cancellous bone body is placed in distilled water as in step (d) under continuous rotation at approximately 80 rpm for approximately 24 hr.

7. The process of claim 1, wherein the cancellous bone body is deproteinized as in step (e) in approximately 4% sodium hypochlorite for approximately 24 hr.

8. The process of claim 1, wherein the processing solution is a buffer.

9. The process of claim 1, wherein the processing solution is phosphate-buffered saline.

10. The process of claim 1, wherein the non-ionic detergent is 1% Triton X-100.

11. The process of claim 1, wherein the DNase and RNase are replaced every 12 hr with freshly prepared DNase and RNase.

12. The process of claim 1, wherein the cancellous bone body treated with DNase and RNase is immersed in phosphate-buffered saline at 37° C. under continuous shaking.

13. The process of claim 1, wherein the decellularized cancellous bone body contains less than approximately 50 ng dsDNA per mg ECM dryweight.

14. The process of claim 1, wherein the decellularized cancellous bone body contains less than approximately 10 ng dsDNA per mg ECM dryweight.

15. The process of claim 1, wherein the decellularized cancellous bone body contains less than 200 bp long DNA fragments.

16. The process of claim 1, wherein the decellularized cancellous bone body does not have visible nuclear material by hematoxylin and eosin staining.

17. The process of claim 1, wherein the lyophilization step utilized is freeze-drying.

18. The process of claim 1, wherein the level of endotoxins present in the decellularized cancellous bone body is less than 0.05 endotoxin units per mL (EU/mL).

19. The process of claim 1, wherein the level of endotoxins present in the decellularized cancellous bone body is less than 0.02 endotoxin units per mL (EU/mL).

20. The process of claim 1, wherein the cryopreservation step utilized is deep freezing.
